(19) United States
(12) Patent Application Publication
(10) Pub. No.: US 2024/0282696 A1
(43) Pub. Date: Aug. 22, 2024

(54) BioFET sensing chip

(71) Applicant: I-CHIH KAO, Hsinchu City (TW)

(72) Inventor: I-CHIH KAO, Hsinchu City (TW)

(21) Appl. No.: 18/582,210

(22) Filed: Feb. 20, 2024

Related U.S. Application Data
(60) Provisional application No. 63/447,216, filed on Feb. 21, 2023.

(51) Int. Cl. H01L 23/522; G01N 27/12; G01N 33/543; H01L 29/78

(52) U.S. Cl. CPC H01L 23/5226; G01N 27/128; G01N 33/54373; H01L 29/78

(57) ABSTRACT

A biological field effect sensing chip is provided, which includes a substrate, a field effect transistor (FET) is disposed on the first region of the substrate, an insulation layer is encapsulated the FET and the first region and disposed on the surface of the second region of the substrate to expose the second region, an interconnect structure is disposed on the second region. The interconnect structure is located in the opening of the insulation layer and the interconnect structure in the opening sequentially includes a bottom conductive layer, an upper conductive layer and a dielectric layer with conductive plug disposed between the bottom conductive layer and the upper conductive layer, and the bottom conductive layer of the interconnect structure is electrically connected to the FET; and a receptor is arranged on the surface of the upper conductive layer to capture the target(s) of the testing sample.

### CROSS-REFERENCE TO RELATED APPLICATIONS

This application claims the benefit of U.S. Provisional Application No. 63/447,216, filed Feb. 21, 2023, which is incorporated in its entirety by reference herein.

### FIELD OF THE INVENTION

The present invention relates to a filed od detection technology, and in particularly to a biological field effect sensing chip that can detect proteins, bacteria, and viruses.

### BACKGROUND OF THE INVENTION

Biosensors are devices that operate based on the electrical, electrochemical, optical and mechanical detection principles and are used to sense and detect biomolecules. Biosensors with transistors can electrically sense charges, photons, and mechanical properties of biomolecules or biological entities. This detection behavior can be achieved through direct detection and induction, or through the reaction or interaction of specific reactants with biomolecules/biological entities. These biosensors can be manufactured by semiconductor processes, which can quickly convert electrical signals, and can be easily applied to integrated circuits (ICs) and microelectromechanical system (MEMs).

Biochip(s) is(are) essentially a miniature laboratory that can perform hundreds or thousands of biochemical reactions simultaneously. Biochip(s) can detect special biomolecules, measure their properties, compute and process signal, and even directly analyze data, so biochip(s) allow researchers to quickly screen large number of biological analytes for purposes ranging from disease diagnosis to detecting biochemical terrorist attacks. Advanced biochip(s) utilize many biosensors alongside fluidic channels for reaction integration, sensing and sample management. BioFET (biological field-effect transistor or bio-organic field-effect transistors) is a biological sensor containing a transistor that can electrically sense biomolecules or biological entities. Although biological field-effect transistor(s) have advantages in many aspects, there are also challenges in their manufacturing and/or operation, such as: issue based on compatibility with semiconductor manufacturing process, biological limitations and/or limits, and there are many challenges that arise in the large-scale integration process, such as the integration of electronic signals and biological applications.

In addition, the existing biosensing chip(s) can only detect the presence/absence of bacteria, viruses or suspended particles, and the range of the detecting region is limited, and the concentration of bacteria, viruses or suspended particles cannot be estimated. In addition, high-sensitivity nanowires designed in a chip-based manner are prone to noise interference, leading misjudgment. Moreover, the nanowires are exposed with polysilicon, which is a special process. However, the most chip factories are unwilling to provide special and customized processes to coordinate production, so the yield cannot be improved, and effective production cannot be achieved.

### SUMMARY OF THE INVENTION

The main object of the present invention is to provide a biological field effect sensing chip, which can be manufactured according to the existing complementary metal oxide semiconductor (CMOS) process of the current semiconductor chip factory.

Another object of the present invention is to arrange the multilayer interconnect structure on the same plane as the field effect transistor and keep the distance apart. In addition, the semiconductor manufacturing process technology is used to electrically connect the bottom conductive layer and the field effect transistor during the process of forming a multilayer interconnect structure, so that no additional manufacturing process is required to simplify the manufacturing process and reduce the costs.

Another object of the present invention is to provide good air tightness between the bottom conductive layer and the isolation layer of the multilayer interconnect structure, so the entire biological field effect sensing chip can be used to detect the sample(s) under liquid state, and there will be no problem of liquid overflowing between the bottom conductive layer and the insulation layer to cause the short-circuit of the biological field effect sensing chip.

According to above objects, the present invention provides a biological field effect sensing chip, including a substrate with a first region and a second region, a field effect transistor arranged on the first region of the substrate, an isolation layer covered the field effect transistor on the first region of the substrate and the second region of the substrate, the isolation layer has an opening to expose the surface of the second region, a multilayer interconnect structure arranged on the surface of the second region of the substrate and disposed in the opening of the isolation layer, and the biological field effect transistor and the multilayer interconnect structure are on the same plane, in which the multilayer interconnect structure in the opening sequentially from the surface of the second region of the substrate to the top includes a bottom conductive layer, an upper conductive layer, and at least a dielectric layer with a plurality of conductive plugs are arranged between the bottom conductive layer and the upper conductive layer, the bottom conductive layer and the upper conductive layer are electrically connected through the plurality of conductive plugs, and the bottom conductive layer of the multilayer interconnect structure is electrically connected to the field effect transistor on the first region of the substrate, and a plurality of receptors arranged on the surface of the upper conductive layer to capture at least one of the plurality of targets in the testing sample.

According to above objects, the present invention also provides another biological field effect sensing chip, which includes a substrate having a source region and a drain region, an isolation layer having an opening on the substrate, and the opening is used to expose a surface of the substrate, a gate oxide layer is disposed on the surface of the substrate, a gate electrode is disposed on the gate oxide layer, a multilayer interconnect structure is disposed on the gate electrode, in which the multilayer interconnect structure includes a bottom conductive layer, an upper conductive layer, and a dielectric layer has a plurality of conductive plugs therein and is disposed between the bottom conductive layer and the upper conductive layer, and the four sides of the upper conductive layer are embedded into the insulation layer to expose the portion of a surface of the upper conductive layer, and a plurality of receptors is arranged on an exposed surface of the upper conductive layer to capture at least one of the plurality of targets in a testing sample.

### BRIEF DESCRIPTION OF THE DRAWINGS

Fig. 1 is a schematic diagram showing a biological field effect sensing chip in accordance with the present invention disclosed herein;

FIG. 2 is a schematic diagram showing a biological field effect sensing chip detecting a testing sample in accordance with the present invention disclosed herein;

FIG. 3 is a schematic diagram showing another embodiment of a biological field effect sensing chip in accordance with the present invention disclosed herein; and

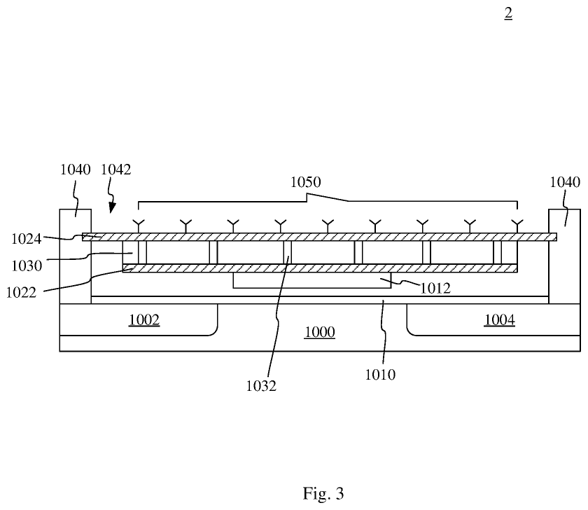

FIG. 4 is a schematic diagram showing another embodiment of a biological field effect sensing chip detecting a testing sample in accordance with the present invention disclosed herein.

### DETAILED DESCRIPTION OF THE PREFERRED EMBODIMENT

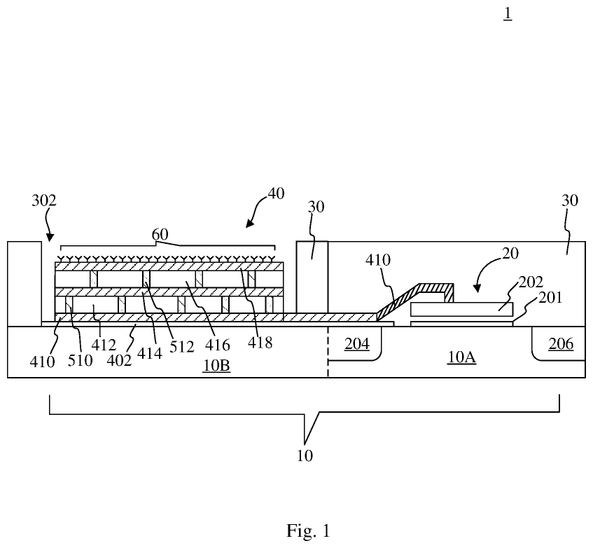

First, please refer to FIG. 1. FIG. 1 shows a cross-sectional schematic diagram of a biological field effect sensing chip. In FIG. 1, a biological field effect sensing chip 1 is composed of a field effect transistor 20 and a multilayer interconnect structure 40, in which the field effect transistor 20 and the multilayer interconnect structure 40 are respectively arranged on a first region 10A and a second region 10B of the substrate 10 and the field effect transistor 20 and the multilayer interconnect structure 40 are arranged on the same plane. It should be noted that, in FIG. 1, the dotted line 110 is used to divide the substrate 10 into the first region 10A and the second region 10B for easy understanding of subsequent descriptions. In fact, there is no dotted line 110 in the substrate 10.

The field effect transistor 20 is an N-type metal oxide semiconductor (NMOS) for example, the structure of the field effect transistor 20 at least includes a gate oxide layer 201, a gate electrode 202, a source region 204 and a drain region 206, in which the source region 204 and the drain region 206 are disposed in the first region 10A of the substrate 10, the gate oxide layer 201 is disposed on the substrate 10, the gate electrode 202 is disposed on the gate oxide layer 201 and is disposed between the source region 204 and the drain region 206. In addition, an isolation layer (or field oxide layer) 30 are further provided on the first region 10A and the second region 10B of the substrate 10. It should be noted that the abovementioned isolation layer 30, the gate oxide layer 201, the gate electrode 202, the source region 204 and the drain region 206 are formed by using a suitable complementary metal oxide semiconductor (CMOS) process, and the formation steps are not the major technology features of this invention, it will not be described herein.

Next, an insulation layer 402 is provided on a surface of the second region 10B of the substrate 10. It should be noted that the insulation layer 402 is formed simultaneously with the gate oxide layer 201 of the field effect transistor 20, and then the portion of the insulation layer 402 is removed by using an etching process. In order to distinction clearly, the insulation layer on the first region 10A is defined as the gate oxide layer 201, and the insulation layer on the second region 10B is defined as the insulation layer 402. Next, a bottom conductive layer 410 is formed on the insulation layer 402 and the bottom conductive layer 410 extends in the direction of the field effect transistor 20 on the same plane and is electrically connected to the gate electrode 202 of the field effect transistor 20. In should be noted that the bottom conductive layer 410 on the second region 10B of the substrate 10 is used as the first metal layer (metal 1) of the multilayer interconnect structure 40, and the bottom conductive layer 410 extends toward the field effect transistor 20 is used as a connecting layer electrically connected to the gate electrode 202. It should be noted that the material of the bottom conductive layer 410 is gold or copper.

Next, an isolation layer 30 is arranged on the substrate 10, in which the isolation layer 30 on the first region 10A of the substrate 10 is used to cover the field effect transistor 20, the isolation layer 30 on the second region 10B of the substrate 10 utilizes an etching process of semiconductor manufacturing process to remove the portion of the insulation layer to form an opening 302 to expose the bottom conductive layer 410 on the second region 10B of the substrate 10. In addition, the isolation layer 30 also covers the portion of the bottom conductive layer 410 extending toward the field effect transistor 20. Next, a first dielectric layer 412 is formed on the bottom conductive layer 410 by using the semiconductor process technology. An etching process is used to remove the portion of the first dielectric layer 412, so that a plurality of first via holes (not shown) is formed in the first dielectric layer 412. Subsequently, the conductive material such as copper, tungsten, titanium, or tantalum or their metal compounds is deposed or electroplated into the plurality of first via holes to form a plurality of first conductive plugs 510.

Next, an intermediate conductive layer 414 is formed on the first dielectric layer 412 having the plurality of first conductive plugs 510, so that the intermediate conductive layer 414 is electrically connected to the bottom conductive layer 410 through the plurality of conductive plugs 510 in the first dielectric layer 412. Then, a second dielectric layer 416 is formed on the intermediate conductive layer 414 by using another deposition process, the manufacturing process is similar to the aforementioned steps of forming a plurality of via holes (not shown), an etching step is used to remove the portion of the second dielectric layer 416 to form a plurality of via holes (not shown) in the second dielectric layer 416. Similarly, the conductive material such as copper, tungsten, titanium, or tantalum or their metal compounds is deposed or electroplated into the plurality of second via holes to form a plurality of second conductive plugs 512. Finally, an upper conductive layer 418 is deposited on the second dielectric layer 416 having the plurality of second conductive plugs 512, in which the upper conductive layer 418 is electrically connected to the intermediate conductive layer 414 through the plurality of second conductive plugs 512. Furthermore, the upper conductive layer 418 and the bottom conductive layer 410 are electrically connected through the plurality of second conductive plugs 512, the intermediate conductive layer 414 and a plurality of first conductive plugs 510.

There is a plurality of receptors 60 is further arranged on the upper conductive layer 418 of the multilayer interconnect structure 40, in which the plurality of receptors 60 is used to capture the target (not shown) in the testing sample (not shown). It should be noted that the above multilayer interconnect structure 40 is composed of the bottom conductive layer 410, the first dielectric layer 412 having a plurality of first conductive plugs 510, the intermediate conductive layer 414, the second dielectric layer 416 with the plurality of second conductive plugs 512, and the upper conductive layer 418. In one embodiment of this invention, the multilayer interconnect structure 40 is composed of the bottom conductive layer 410, the upper conductive layer 418 and the first dielectric layer 412 with the plurality of first conductive plugs 510 between the bottom conductive layer 410 and the upper conductive layer 418. In another embodiment of this invention, the multilayer interconnect structure 40 is formed by a stagged stack of four, five or more conductive layers and the dielectric layer having the plurality of conductive plugs between each the conducive layers. However, no matter how many layers of conductive layers and the dielectric layer are interlaced, the multilayer interconnect structure 40 is electrically connected through the bottom conductive layer 410 extending toward the gate electrode 202 of the field effect transistor 20 and serving as a connecting layer, the surface of the uppermost layer of the multilayer interconnect structure 40 is the surface of the upper conductive layer 418, the surface of the upper conductive layer 418 is arranged a plurality of receptors 60 to capture at least one of the plurality of targets (not shown) in the testing sample (not shown), in which the plurality of receptors 60 is fixed on the upper conductive layer 418 by an immobilized method during the antibody processing after the production of the biological field effect sensing chip 1. Accordingly, in this invention, the multilayer interconnect structure 40 having a plurality of receptors 60 on the second region 10B of the substrate 10 is defined as a detecting region or sensing region.

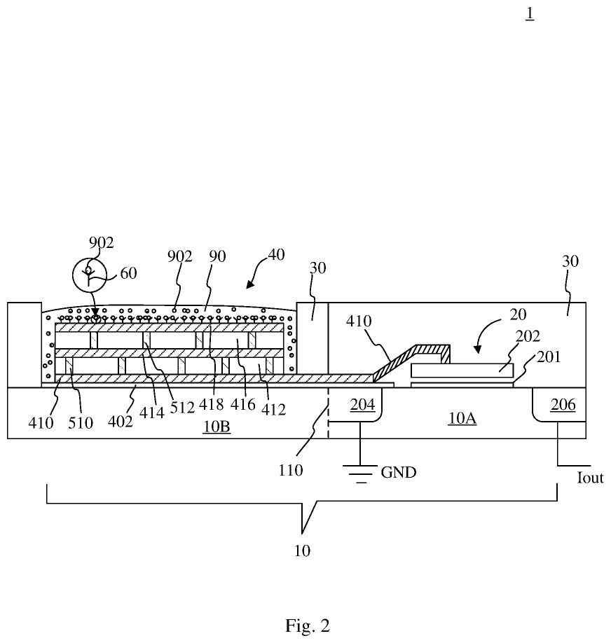

Please continue to refer to FIG. 2. FIG. 2 is a schematic diagram showing a biological field effect sensing chip for detecting a testing sample according to the technology disclosed in the present invention. In FIG. 2, when the testing sample 90 with a plurality of targets (or target molecules) 902 is placed in the detecting region, thereby the testing sample is allowed to fully contact the plurality of receptors 60 for a period time. During the reaction process, the plurality of receptors 60 on the upper conductive layer 418 is used to capture at least one of the plurality of targets (or target molecules) 902 in the testing sample 90, after the plurality of receptors 60 captured the at least one of the plurality of targets (or target molecules) 902, the voltage value is transmitted to the gate electrode 202 of the field effect transistor 20 through the upper conductive layer 418, the plurality of second conductive plugs 512, the intermediate conductive layer 414 and the plurality of first conductive plugs 510, and then the gate electrode 202 of the field effect transistor 20 outputs the current value (Iₒᵤₜ) which is corresponding to the voltage value to an external processing unit (not shown) to obtain the concentration value (or quantity) of the plurality of targets 902 in the testing sample 90.

For example, the testing sample 90 includes a BTP buffer containing unknown target concentration, whole blood or plasma, in which when the testing sample 90 is whole blood or plasma, the testing sample 90 is diluted with BTP buffer solution. Next, the diluted testing sample 90 is dropped into the detecting region (that is, the second region 10B of the substrate), the diluted testing sample 90 is allowed to stand for a period of time, so as to the plurality of receptors 60 is fully contacted the diluted testing sample 90, so that the plurality of receptors 60 has enough time to capture at least one of the plurality of targets 902 in the diluted testing sample 90. The voltage value of the upper conductive layer 418 will change with the number of the targets 902 in the diluted testing sample 90 captured by the plurality of receptors 60, and the field effect transistor 20 will output the voltage value in the form of current (Iₒᵤₜ in FIG. 2) that is corresponding to the upper conductive layer 418 to the external processing unit (not shown) which is connected to the biological field effect sensing chip 1, thereby, the external processing unit (not shown) is provided for processing the current change generated by the plurality of receptors 60 capturing the plurality of targets 902 in the diluted testing sample 90, so the concentration value (or quantity) of the plurality of targets 902 in the testing sample 90 can be obtained. In this embodiment, the targets 902 in the testing sample 90 can be an organism. When the testing sample 90 is buffer solution, the targets 902 in the buffer solution includes yeast, bacteria, viruses or proteins. When the testing sample 90 is plasma, the targets 902 in the plasma is cell.

According to abovementioned, since the good air tightness between the bottom conductive layer 410 and the isolation layer 30 of the multilayer interconnect structure 40 disclosed in this invention, when the testing sample is plasma, whole blood or other testing sample in liquid state that is dropped into the opening 302 (the second region 10B where the multilayer interconnect structure 40 is located). The testing sample 90 in liquid state is not overflowed between the bottom conductive layer 410 and the isolation layer 30 of the multilayer interconnect structure 40 and the short of the field effect transistor 20 is not induced, so that the durability of the biological field effect sensing chip 1 is improved. Accordingly, the multilayer interconnect structure 40 and the field effect transistor 20 are integrated on the substrate 10 to form the biological field effect sensing chip 1 which can achieve the purpose of the liquid detection on the chip.

The present invention also provides another embodiment of the biological field effect sensing chip 2, which includes a substrate 1000 having a source region 1002 and a drain region 1004 therein, an isolation layer 1040 having an opening 1042 on the substrate 10 and the opening 1042 is used to expose the surface of the substrate 1000, a gate oxide layer 1010 is disposed on the surface of the substrate 1000, a gate electrode 1012 is disposed on the gate oxide layer 1010, a multiplayer interconnect structure is arranged on the gate electrode 1012, in which the multilayer interconnect structure includes a bottom conductive layer 1022, the upper conductive layer 1024, and a dielectric layer 1030 with a plurality of conductive plugs 1030 between the bottom conductive layer 1022 and the upper conductive layer 1024, and the four sides of the upper conductive layer are embedded into the insulation layer 1040 to expose the portion of a surface of the upper conductive layer 1024, a plurality of receptors 1050 is arranged on the exposed surface of the upper conductive layer 1024 to capture at least one of the plurality of targets (not shown) in a testing sample (not shown). It should be noted that where the region of the plurality of receptors 1050 is regarded as the detecting region. In this embodiment, the material, structure, and/or the function of the multilayer interconnect structure is same as the abovementioned in FIG. 1 and FIG. 2, thus, it is not to describe repeatedly herein.

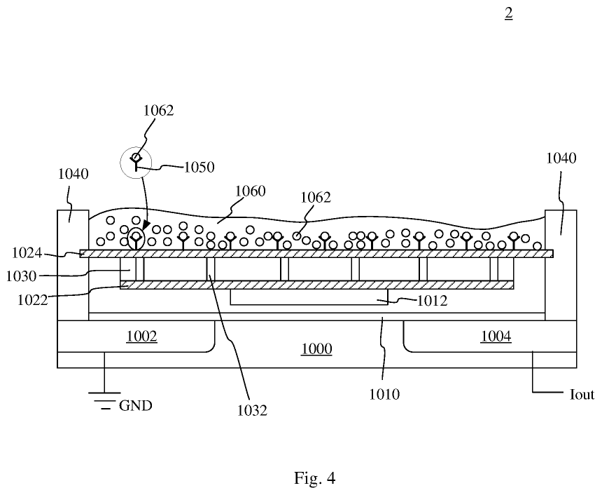

Next, please refer to FIG. 4. FIG. 4 is a schematic diagram showing another embodiment of a biological field effect sensing chip detecting a testing sample according to the technology disclosed in the present invention. In FIG. 4, when the testing sample 1060 with a plurality of targets (or target molecules) 1062 is dropped into the detecting region of the biological field effect sensing chip 2, the testing sample 1060 is allowed to fully contact the plurality of receptors 1050 for a period of time. During the reaction process, the plurality of receptors 1050 on the surface of the upper conductive layer 1024 is used to capture the plurality of targets (or target molecules) 1062 in the testing sample 1060. Similarly, after the plurality of receptors 1050 captured the at least one of the plurality of targets (or target molecules) 1062 in the testing sample 1060, the voltage value is transmitted to the gate electrode 1012 through the upper conductive layer 418, the plurality of conductive plugs 1032 and the bottom conductive layer 1022 and outputs the current value (Iₒᵤₜ) which is corresponding to the voltage value to an external processing unit (not shown) through the drain region 1004 to obtain the concentration value (or quantity) of the plurality of targets 1062 in the testing sample 1060.

In this invention, it should be noted that the four sides of the upper conductive layer 1024 of the multilayer interconnect structure are embedded into the isolation layer 1040, thereby, there is no void or gap between the upper conductive layer 1024 and the isolation layer 1040 to obtain the good air tightness between the upper conductive layer 1024 and the isolation 1024, so as to the biological field effect sensing chip 2 also has good air tightness to prevent the leakage or overflow when the testing sample 1060 is dropped into the biological field effect sensing chip 2. In addition, the upper conductive layer 1024 can be regarded as an entire metal layer that is placed on the uppermost layer of the multilayer interconnect structure. Accordingly, when the plurality of receptors 1050 is fixed on the surface of the upper conductive layer 1024 can be formed by spin on method, so the more receptors 1050 are allowed to form on the upper conductive layer 1024, and the shape of the upper conductive layer 1024 is not to be considered.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A biological field effect sensing chip, comprising:
a substrate having a first region and a second region;
a filed effect transistor, which is arranged on the first region of the substrate;
an isolation layer, which is covered the field effect transistor on the first region of the substrate and is arranged on the second region of the substrate, and has an opening to expose a surface of the second region of the substrate;
a multilayer interconnect structure, which is arranged on the surface of the second region of the substrate and is disposed in the opening of the isolation layer, the biological field effect transistor and the multilayer interconnect structure are on the same plane, wherein the multilayer interconnect structure in the opening sequentially from the surface of the second region of the substrate upward includes:
a bottom conductive layer, which is disposed on the surface of the second region of the substrate; and
an upper conductive layer, which is disposed on the bottom conductive layer, wherein a dielectric with a plurality of conductive plugs is arranged between the bottom conductive layer and the upper conductive layer, the bottom conductive layer and the upper conductive layer are electrically connected through the plurality of conductive plugs, and the bottom conductive layer of the multilayer interconnect structure is electrically connected to the field effect transistor on the first region of the substrate; and
a plurality of receptors, which is arranged on a surface of the upper conductive layer to capture at least one of plurality of targets in a testing sample.

2. The biological field effect sensing chip according to claim 1, wherein the field effect transistor includes a gate oxide layer, a gate electrode, a source region and a drain region, the source region and the drain region is disposed in the substate, and the gate oxide layer is disposed on the substrate and the gate electrode is disposed on the gate electrode and is located between the source region and drain region.

3. The biological field effect sensing chip according to claim 1, wherein the multilayer interconnect structure is electrically connected to the gate electrode of the field effect transistor from the bottom conductive layer through the insulation layer.

4. The biological field effect sensing chip according to claim 1, wherein the bottom conductive layer is gold or copper.

5. The biological field effect sensing chip according to claim 1, further comprising an insulation layer on the surface of the second region of the substrate.

6. The biological field effect sensing chip according to claim 1, further comprising an external processing unit is electrically connected to the biological field effect sensing chip for processing a value corresponding to a current change generated when the plurality of receptors captures at least one of the plurality of targets in the testing sample.

7. The biological field effect sensing chip according to claim 1, wherein the target includes yeast, bacteria, cells, viruses, proteins, DNA or RNA.

8. A biological field effect sensing chip, comprising:
a substrate having a source region and a drain region therein;
an insulation layer having an opening on the substrate, and the opening is used to expose a surface of the substrate;
a gate oxide layer, which is disposed on the surface of the substrate;
a gate electrode, which is disposed on the gate oxide layer;
a multilayer interconnect structure, which is disposed on the gate electrode, wherein the multilayer interconnect structure includes a bottom conductive layer, an upper conductive layer, and a dielectric layer has a plurality of conductive plugs therein and is disposed between the bottom conductive layer and the upper conductive layer, and the four sides of the upper conductive layer are embedded into the insulation layer to expose the portion of a surface of the upper conductive layer; and
a plurality of receptors, which is arranged on an exposed surface of the upper conductive layer to capture at least one of a plurality of targets in a testing sample.

9. The biological field effect sensing chip according to claim 8, wherein the bottom conductive layer and the upper conductive layer is metal.

10. The biological field effect sensing chip according to claim 9, wherein the metal includes gold, copper, or aluminum.

11. The biological field effect sensing chip according to claim 8, further comprising an external processing unit is electrically connected to the biological field effect sensing chip for processing a value corresponding to a current change generated when the plurality of receptors captures at least one of the plurality of targets in the testing sample.

12. The biological field effect sensing chip according to claim 8, wherein the target includes yeast, bacteria, cells, viruses, proteins, DNA or RNA.
